(19) United States
(12) Patent Application Publication
(10) Pub. No.: US 2026/0216370 A1
(43) Pub. Date: Jul. 30, 2026

(54) RETINA-TARGETING ADENO-ASSOCIATED VIRUS VARIANT ADN AND USE THEREOF

(71) Applicant: UIF (University Industry Foundation), Yonsei University, Seoul (KR)

(72) Inventors: Jae-Hyung JANG, Seoul (KR); Junwon LEE, Seoul (KR); Jinyoung KIM, Seoul (KR); Subin CHO, Seoul (KR); Han JEONG, Seoul (KR)

(73) Assignee: UIF (University Industry Foundation), Yonsei University, Seoul (KR)

(21) Appl. No.: 19/457,608

(22) Filed: Jan. 23, 2026

Related U.S. Application Data
(60) Provisional application No. 63/749,122, filed on Jan. 24, 2025.

(30) Foreign Application Priority Data
Aug. 13, 2025 (KR) 10-2025-0112552

(51) Int. Cl. A61K 48/00; A61K 9/00; C07K 14/005; C12N 15/86

(52) U.S. Cl. CPC A61K 48/0041; A61K 9/0048; A61K 48/0075; C07K 14/005; C12N 15/86; C12N 2750/14122; C12N 2750/14143; C12N 2750/14145

(57) ABSTRACT

Disclosed is an adeno-associated virus (AAV) capsid protein variant having improved infectivity and transduction efficiency into target cells, and uses thereof. Specifically, disclosed is an AAV2 variant capsid protein selected by directed evolution for gene delivery into retinal cells, a recombinant AAV vector including the same, and uses thereof as a gene delivery vehicle for retinal cells.

### CROSS-REFERENCE TO RELATED APPLICATION

This application is based on and claims priority under 35 U.S.C. § 119 to U.S. Provisional Application No. 63/749,122, filed on Jan. 24, 2025, and to Korean Patent Application No. 10-2025-0112552, filed on Aug. 13, 2025, the disclosures of which are incorporated by reference herein in their entirety.

### INCORPORATION BY REFERENCE OF SEQUENCE LISTING

The content of the electronically submitted sequence listing, file name: Q317625SEQLIST-ST26.xml; size: 7,496 bytes; and date of creation: Jan. 23, 2026, filed herewith, is incorporated herein by reference in its entirety.

### BACKGROUND

### 1. Field

The disclosure relates to an adeno-associated virus (AAV) capsid protein variant having improved infectivity and transduction efficiency into target cells, and uses thereof. Specifically, the disclosure relates to an AAV2 variant capsid protein selected for gene delivery into retinal cells through design and validation using an AI-based model, a recombinant AAV vector including the same, and uses thereof as a gene delivery vehicle for retinal cells.

This research was conducted with the support of the Samsung Science & Technology Foundation (Project No. SRFC-MA2022-08).

### 2. Description of the Related Art

Gene therapy is a method of treating hereditary diseases by introducing a normal gene into human cells to correct or compensate for diseases caused by genetic defects and abnormalities, and has become a key means of treating various hereditary diseases.

Adeno-associated virus (AAV) is a gene delivery vector widely used in the field of gene therapy because it may infect a wide range of tissues, is non-pathogenic, and has low immunogenicity. AAV exists in various serotypes, and depending on the serotype, characteristics such as infection host or tissue specificity, transmissibility, and gene transduction efficiency differ. For example, serotype 2 (AAV2) may infect various cells, and serotype 1 (AAV1), serotype 5 (AAV5), and serotype 6 (AAV6) have higher tissue specificity than AAV2, and it has been reported that AAV1 has high gene transduction efficiency into muscle, liver, the airway, and the central nervous system, AAV5 has high gene transduction efficiency into the central nervous system, liver, and retina, and AAV6 has high gene transduction efficiency into the heart, muscle, liver, etc.

However, to be utilized as a gene delivery vector for gene therapy, it is necessary to have higher tissue specificity and gene delivery and expression efficiency than existing AAV vectors. To this end, AAV capsid engineering that modifies the sequence of the AAV capsid protein has been actively studied.

Directed evolution is a representative high-throughput screening technique for improving the function of biomolecules, and is a method that mimics the process of natural selection by inducing genetic mutation and repeating selection processes. AAV capsid directed evolution is conducted by introducing mutations into a wild-type AAV capsid gene to construct an AAV capsid library of diverse sequences, and then screening this library to identify and obtain capsid variants having desired characteristics. However, there is a limitation in that an enormous amount of time and cost are required for the preparation and screening of numerous variant candidates.

Accordingly, in recent years, studies utilizing artificial intelligence (AI) have been actively conducted in the field of AAV capsid engineering, and methods have been utilized in which promising capsid variant candidates are selected in silico through AI-based predictive models, and then verified through actual experiments to obtain capsid variants.

Through AAV capsid engineering, recombinant AAV vectors with improved tissue specificity and gene transduction efficiency have been developed and applied to various indications (for example, Korean Registered U.S. Pat. No. 2,234,930). In particular, hereditary retinal diseases often result from single-gene abnormalities and thus have attracted attention as promising indications for gene therapy, and for this purpose, variants such as AAV2-7m8, AAV2.GL, and AAV2.NN have been developed and utilized for gene delivery into the retina.

However, there is still a demand for AAV variants having excellent transduction capability capable of inducing highly specific delivery and gene expression in specific cells within the retina.

Accordingly, the inventors of the disclosure constructed an artificial intelligence-based predictive model to obtain AAV capsid variants that were not experimentally discovered using an AAV capsid screening method utilizing a random library, and completed the disclosure by designing and verifying, using the same, AAV capsid variants having excellent gene transduction efficiency into target cells of the retina.

### SUMMARY

Provided is an AAV2 variant capsid protein having gene delivery capability to retinal cells through intravitreal injection.

In addition, provided is a recombinant AAV2 vector including an AAV2 variant capsid protein having gene delivery capability to retinal cells through intravitreal injection.

Furthermore, provided is a use of a recombinant AAV2 vector including an AAV2 variant capsid protein having gene delivery capability to retinal cells through intravitreal injection for treatment of ocular diseases.

Additional aspects will be set forth in part in the description, which follows and, in part, will be apparent from the description, or may be learned by practice of the presented embodiments of the disclosure.

According to an aspect of the disclosure, provided is an AAV2 variant capsid protein including a peptide NAPPKNP inserted into a GH-loop of a wild-type AAV2 capsid protein.

The inventors of the disclosure obtained the AAV2 variant capsid protein of the disclosure by selecting and validating variants of an AAV2 capsid protein using artificial intelligence, to identify AAV capsid sequences that had not been experimentally identified through AAV screening using a random library.

Specifically, data on variant sequences of a wild-type AAV2 capsid protein selected in human retinal organoids and their gene delivery capability to photoreceptor cells were secured, and based on these data, artificial intelligence was used to train correlations between sequences and gene delivery capability to photoreceptor cells, and based thereon, a model was constructed to predict sequences of AAV2 capsid variants predicted to have excellent gene delivery capability to photoreceptor cells. Using the prediction model constructed in this way, candidate AAV2 capsid variant sequences were designed, and gene delivery activity was verified by applying the same to actual photoreceptor cells, thereby obtaining the AAV2 variant capsid protein of the disclosure.

According to an aspect of the disclosure, the peptide NAPPKNP may be inserted at amino acid 587 of the wild-type AAV2 capsid protein of SEQ ID NO: 1 or at a position corresponding thereto.

The AAV2 variant capsid protein of the disclosure includes an insertion of NAPPKNP between amino acid N587 and amino acid R588 of the wild-type AAV2 capsid protein of SEQ ID NO: 1, and was confirmed to transduce retinal cells including photoreceptor cells, ganglion cells, Müller glial cells, and bipolar cells by intravitreal injection to deliver a target gene at high efficiency, as compared with wild-type AAV2 and AAV2.7m8 (having insertion of the peptide “LGETTRP” between N587 and R588 of wild-type AAV2: SEQ ID NO: 3) and AAV2.NN (having insertion of the peptide “NNPTPSR” between N587 and R588 of wild-type AAV2: SEQ ID NO: 4), which are known to have gene delivery capability to retinal photoreceptor cells, and was named “AAV2.ADN” or an “ADN variant.”

The ADN variant according to the disclosure was confirmed to pass through an inner limiting membrane (ILM) in the retina by intravitreal injection, deliver a target gene to photoreceptor cells and induce strong gene expression, and result in delivery to deeper cells through retinal layers and increased expression of a target gene as compared with wild-type AAV2, and was confirmed to have excellent gene transduction efficiency through increased gene expression and photoreceptor cell-specific gene delivery as compared with wild-type AAV2 in human retinal organoids.

The wild-type AAV2 capsid protein includes the amino acid sequence of SEQ ID NO: 1. The ADN variant according to the disclosure includes NAPPKNP between N587 and R588 among the amino acid sequence of SEQ ID NO: 1.

According to an aspect of the disclosure, the AAV2 variant capsid protein may be composed of the amino acid sequence of SEQ ID NO: 2 or an amino acid sequence having 85% or more, 90% or more, or 95% or more sequence identity thereto.

As used herein, the term “AAV” is an abbreviation of adeno-associated virus, and refers to the virus itself or a derivative thereof. Unless otherwise specified, AAV includes all subtypes thereof, naturally occurring forms, and recombinant forms thereof. AAV is a non-pathogenic parvovirus consisting of a single-stranded DNA genome of 4.7 kb in length within a non-enveloped icosahedral capsid. The genome includes three open reading frames (ORFs) flanked by inverted terminal repeats (ITRs) that function as viral origins of replication and packaging signals the rep ORF encodes four non-structural proteins that play roles in viral replication, transcriptional regulation, site-specific integration, and virion assembly, and the cap ORF encodes three structural proteins (VP1-3) that assemble to form a 60-mer viral capsid, and an ORF present as an alternative reading frame within the cap gene encodes an assembly-activating protein (AAP), which is a viral protein that localizes AAV capsid proteins to the nucleolus and functions in the capsid assembly process.

The genome sequences and native terminal repeat (terminal repeat: TR) sequences of various serotypes of AAV, and Rep proteins and capsid subunits thereof, are known in the art. These sequences may be found in the literature or in public databases such as GenBank. For example, the genome sequence of AAV2 is registered under GenBank Accession NC_001401.

As used herein, the term “tropism” refers to preferential targeting of cells of a particular host species or a particular cell type within a host species by a virus (for example, AAV), and is used interchangeably with “taxis.” For example, a virus that may infect retinal cells with high specificity has a higher tropism for retinal cells than for non-retinal cells. Since the capsid protein of an AAV virus determines infectivity toward target cells or tissues, tropism or taxis are used as a selection pressure to develop AAV variants that may deliver genes to target cells or tissues with high specificity and efficiency through capsid engineering via directed evolution.

As used herein, the term “retinal cells” refers to all types of cells present in the retina, and include, for example, photoreceptor cells, retinal ganglion cells, bipolar cells, Müller glial cells, horizontal cells, amacrine cells, astrocytes, microglia, retinal vascular cells, and retinal pigment epithelial cells (retinal pigment epithelium: RPE), but are not limited thereto.

As used herein, the term “gene transduction efficacy” refers to the ability or activity of an AAV vector to specifically deliver and express a gene in target cells or tissues, and is used interchangeably herein with “infectivity” or “transduction efficacy.”

As used herein, the term “corresponding position” refers to a position corresponding through sequence alignment with respect to a specific position, or a position determined to perform the same or substantially the same function.

As used herein, the term “sequence identity” refers to a ratio at which the type of amino acid or base at each specific position matches between polypeptides or polynucleotides being compared, and may be used interchangeably with “homology” with respect to sequences. For example, calculation of homology (%) between two polypeptide sequences is performed by optimally aligning the two sequences for comparison purposes and comparing amino acids at corresponding amino acid positions, and if the same amino acid is present at a position of the first sequence and the corresponding position of the second sequence, the two sequences are determined to be identical at that position. Sequence homology may be determined using software such as BLASTP, BLASTN, and FASTA.

According to an aspect of the disclosure, the AAV2 variant capsid protein may confer increased infectivity to retinal cells as compared with a wild-type AAV2 capsid protein.

According to an aspect of the disclosure, the retinal cells may be selected from the group consisting of photoreceptor cells, retinal ganglion cells, bipolar cells, Müller glial cells, horizontal cells, amacrine cells, astrocytes, microglia, retinal vascular cells, and retinal pigment epithelial cells.

According to an aspect of the disclosure, the AAV2 variant capsid protein may confer increased infectivity to photoreceptor cells by intravitreal injection as compared with a wild-type AAV2 capsid protein.

According to another aspect of the disclosure, provided is an isolated nucleic acid encoding an AAV variant capsid protein.

According to an aspect of the disclosure, the isolated nucleic acid may include a nucleotide sequence encoding the amino acid sequence of SEQ ID NO: 2 or a nucleotide sequence having 95% or more sequence homology thereto.

According to another aspect of the disclosure, provided is a host cell including an isolated nucleic acid encoding an AAV2 variant capsid protein including a peptide NAPPKNP inserted at amino acid 587 of a wild-type AAV2 capsid protein of SEQ ID NO: 1 or at a position corresponding thereto.

The host cell may be used to produce a recombinant AAV virion including an AAV2 variant capsid protein. In this case, the host cell is referred to as a “packaging cell.” The host cell may be selected from various cells including, but not limited to, HEK293 cells, HEK293T cells, Hela cells, Sf9 cells, 293A cells, 293H cells, etc.

According to an aspect of the disclosure, the isolated nucleic acid is stably or transiently introduced into a host cell using methods including, but not limited to, electroporation, calcium phosphate precipitation, liposome-mediated transfection, and the like.

According to another aspect of the disclosure, provided is an AAV2 variant capsid protein including a peptide NAPPKNP inserted at amino acid 587 of a wild-type AAV2 capsid protein of SEQ ID NO: 1 or at a position corresponding thereto, and

a recombinant AAV vector including a heterologous nucleic acid including a sequence encoding a target product.

As used herein, the term “recombinant AAV vector” refers to an AAV vector including a polynucleotide sequence that is not derived from AAV, that is, a polynucleotide sequence that is heterologous with respect to AAV, and is used interchangeably with “rAAV.” Typically, in a recombinant AAV vector, the heterologous polynucleotide sequence is a nucleic acid sequence encoding a target product to be delivered to a target cell.

As used herein, the term “heterologous” refers to a source that is genetically distinct from an AAV2 vector, for example, a nucleic acid originating from a different species.

According to an aspect of the disclosure, the recombinant AAV vector may exhibit increased transduction into retinal tissues/cells as compared with an AAV vector (AAV2 vector) including a wild-type AAV2 capsid protein.

According to an aspect of the disclosure, the recombinant AAV vector may exhibit increased transduction into retinal tissues/cells through intravitreal injection as compared with an AAV vector (AAV2 vector) including a wild-type AAV2 capsid protein.

The recombinant AAV vector according to an embodiment may be used to prepare a recombinant AAV virion. When introduced into a suitable host cell, the recombinant AAV vector may provide production of a corresponding recombinant AAV virion.

According to an aspect of the disclosure, the target product may be a polypeptide or a nucleic acid. The target product refers to a product that may supplement or enhancing a function that is deficient or insufficient in a subject, or reducing or eliminating a defective function, thereby may bring about a therapeutic effect for a target disease. For example, the target product may be a gene product or its activity that is directly or indirectly related to an ocular disease such as a retinal disease, or may be one that enhances the function of retinal cells, and may be, for example, pigment epithelium-derived factor (PEDF), or rhodopsin.

According to an aspect of the disclosure, the target product may be a therapeutic nucleic acid and may target any gene associated with an ocular disease, such as achromatopsia, macular degeneration such as wet age-related macular degeneration (AMD) or dry AMD, cataracts, pan choroidal atrophy, glaucoma, optic neuropathy, Marfan syndrome, myopia, nodular choroidal angiopathy, retinitis pigmentosa, Stargardt disease, Usher syndrome, Leber congenital amaurosis, Leber hereditary optic neuropathy, uveal melanoma, or X-linked retinoschisis or any hereditary disease occurring in ocular tissue. The target gene may be, a gene encoding a protein selected from the group consisting of, for example, VEGFA, VEGFB, ANGPT2, FLT1, KDR, PDGFB, CFH, ARMS2, HTRA1, TIMP3, C3, C2, CFB, SERPINF1, ANGPT1, CFI, CD59, ABCA1, ABCA4, RHO, RPGR, PRPF3, PRPF31, PRPF8, PDE6A, PDE6B, PDE6G, CNGB1, CNGA1, AIPL1, CRX, CRB1, CEP290, RS1, USH2A, MYO7A, USH1C, CLRN1, BBS1, BBS10, BEST1, PROM1, ELOVL4, PRPH2, RDH12, RPE65, CDH3, PCDH15, GUCY2D, GUCA1A, TULP1, LRAT, GNAT2, CNGB3, CNGA3, PDE6C, PDE6H, ATF6, GRM6, GPR179, TRPM1, NYX, CABP4, NR2E3, NRL, IMPDH1, IMPDH2, SNRNP200, HK1, GNAT1, KCNV2, RAX2, CHM, MERTK, EYS, NMNAT1, RP1, RP2, LCA5, RD3, RLBP1, MT-ND4, MT-ND1, MT-ND6, NDP, LRP5, TSPAN12, FZD4, CAPN5, TEK, CYP1B1, CYP4V2, MYOC, OPTN, OPA1, IL10, IL1RN, TNFRSF1A, HGF, PEDF, sFLT1, STNFR, GNAQ, GNA11, BAP1, SF3B1, EIF1AX, TP53, TNFSF10, DDEF1, CDKN2A, p14ARF, IFNB1, HSV-TK, BDNF, GDNF, CNTF, NTF3, NGF, XIAP, SOD2, TXN, NAMPT, SLC16A8, GEMIN4, CYP51A1, RIC1, TAPT1, TAFIA, WDR87, APE1, MIP, GJA3, GJA8, CRYAA, CRYBB2, PRX, POLR3B, XRCC1, ZNF350, EPHA2, REP1, CALM2, MPP7, LOX1, CAV1, CAV2, PITX2, FOXC1, PAX6, LTBP2, FBN1, TGFBR2, MTHFR, MTR, MTRR, MET, UMODL1, MMP1, MMP2, CBS, IGF1, UHRF1BP1L, PTPRR, PPFIA2, P4HA2, SERPING1, FGD6, ABCG1, LOC387715, CETP, PTEN, and HERC2/OCA2, but is not limited thereto.

According to an aspect of the disclosure, the target product may be a therapeutic protein or polypeptide, and may be selected from the group consisting of, for example, VEGFA, VEGFB, ANGPT2, FLT1, KDR, PDGFB, CFH, ARMS2, HTRA1, TIMP3, C3, C2, CFB, SERPINF1, ANGPT1, CFI, CD59, ABCA1, ABCA4, RHO, RPGR, PRPF3, PRPF31, PRPF8, PDE6A, PDE6B, PDE6G, CNGB1, CNGA1, AIPL1, CRX, CRB1, CEP290, RS1, USH2A, MYO7A, USH1C, CLRN1, BBS1, BBS10, BEST1, PROM1, ELOVL4, PRPH2, RDH12, RPE65, CDH3, PCDH15, GUCY2D, GUCA1A, TULP1, LRAT, GNAT2, CNGB3, CNGA3, PDE6C, PDE6H, ATF6, GRM6, GPR179, TRPM1, NYX, CABP4, NR2E3, NRL, IMPDH1, IMPDH2, SNRNP200, HK1, GNAT1, KCNV2, RAX2, CHM, MERTK, EYS, NMNAT1, RP1, RP2, LCA5, RD3, RLBP1, MT-ND4, MT-ND1, MT-ND6, NDP, LRP5, TSPAN12, FZD4, CAPN5, TEK, CYP1B1, CYP4V2, MYOC, OPTN, OPA1, IL10, IL1RN, TNFRSF1A, HGF, PEDF, sFLT1, STNFR, GNAQ, GNA11, BAP1, SF3B1, EIF1AX, TP53, TNFSF10, DDEF1, CDKN2A, p14ARF, IFNB1, HSV-TK, BDNF, GDNF, CNTF, NTF3, NGF, XIAP, SOD2, TXN, NAMPT, SLC16A8, GEMIN4, CYP51A1, RIC1, TAPT1, TAFIA, WDR87, APE1, MIP, GJA3, GJA8, CRYAA, CRYBB2, PRX, POLR3B, XRCC1, ZNF350, EPHA2, REP1, CALM2, MPP7, LOX1, CAV1, CAV2, PITX2, FOXC1, PAX6, LTBP2, FBN1, TGFBR2, MTHFR, MTR, MTRR, MET, UMODL1, MMP1, MMP2, CBS, IGF1, UHRF1BP1L, PTPRR, PPFIA2, P4HA2, SERPING1, FGD6, ABCG1, LOC387715, CETP, PTEN, and HERC2/OCA2, but is not limited thereto.

According to another aspect of the disclosure, provided is a use of the above-described recombinant AAV vector in the treatment of an ocular disease.

According to an aspect of the disclosure, provided is a pharmaceutical composition for treatment of an ocular disease, including the above-described recombinant AAV vector.

According to an aspect of the disclosure, provided is a method of treating an ocular disease, including administering a therapeutically effective amount of the above-described recombinant AAV vector to a subject in need of treatment of an ocular disease.

As used herein, the term “ocular disease” refers to a disease related to the eye or a part or specific region of the eye, for example, the retina or choroid.

According to an aspect of the disclosure, the ocular disease may be a retinal disease, a choroidal disease, or a retinal/choroidal disease.

According to an aspect of the disclosure, the ocular disease may be selected from retinal vascular disease, optic neuropathy, hereditary retinal disease, hereditary choroidal disease, and intraocular tumor.

According to an aspect of the disclosure, the ocular disease may be selected from retina-vitreous-choroid related diseases, retinal vascular diseases, macular diseases, hereditary retinal diseases, hereditary vitreous diseases, hereditary choroidal diseases, intraocular inflammatory diseases, intraocular tumors, glaucoma, and optic neuropathies.

According to an aspect of the disclosure, the ocular disease may be, occlusion of retinal vessels including age-related macular degeneration, diabetic retinopathy, retinal artery/vein occlusion, and macular telangiectasia, optic neuropathy including glaucoma, hereditary retinal/choroidal diseases including uveitis, Leber congenital amaurosis, retinitis pigmentosa, Usher syndrome, Stargardt's Disease, Best's Disease, X-linked retinoschisis, congenital stationary night blindness, choroideremia, achromatopsia, cone dystrophy, gyrate atrophy, and Bardet-Biedl syndrome, and intraocular tumors such as uveal melanoma, intraocular lymphoma, and retinoblastoma, but are not limited thereto.

According to an aspect of the disclosure, the ocular disease may be selected from macular degeneration, diabetic retinopathy, occlusion of retinal vessels, macular telangiectasia, retinopathy of prematurity, myopic degeneration, retinitis pigmentosa, Leber congenital amaurosis, Best's Disease, Stargardt's Disease, congenital stationary night blindness, X-linked retinoschisis, Bietti crystalline dystrophy, achromatopsia, cone dystrophy, cone-rod dystrophy, maculopathy, Usher syndrome, Bardet-Biedl syndrome and other syndromic retinitis pigmentosa, pan choroidal atrophy, central areolar choroidal dystrophy, gyrate atrophy of choroid and retina, glaucoma, other optic neuropathies, uveitis, uveal melanoma, intraocular lymphoma, retinoblastoma, retinoschisis, and other retinal injury.

According to an aspect of the disclosure, the recombinant AAV vector may be administered by intravitreal injection, subretinal injection, intraocular injection through suprachoroidal injection, or any suitable route of administration for delivery to the retina, including, but not limited to, periocular, intravenous, intraarterial, intranasal administration, etc.

According to an aspect of the disclosure, the recombinant AAV vector may be administered by intravitreal injection. The recombinant AAV vector of the disclosure, when administered via intravitreal injection, may migrate through the vitreous humor, pass through the ILM, and migrate through the retinal layers more efficiently than an AAV vector including a wild-type AAV2 capsid protein.

According to an aspect of the disclosure, the recombinant AAV vector may be administered by intraocular injection.

The recombinant AAV vector according to the disclosure has an increased ability, when administered by intravitreal injection, to deliver and express a heterologous nucleic acid to retinal cells across the ILM as compared with an AAV2 vector including a wild-type capsid protein.

The recombinant AAV vector according to the disclosure may cross the ILM and may traverse cell layers including Müller glial cells, bipolar cells, etc. to reach photoreceptor cells. For example, when administered via intravitreal injection, the recombinant AAV vector according to the disclosure may cross the ILM and may traverse cell layers including Müller glial cells, bipolar cells, etc. to reach photoreceptor cells.

In some embodiments, the recombinant AAV vector may specifically infect retinal cells, and in particular, may specifically infect photoreceptor cells.

As used herein, the term “therapeutically effective amount” refers to an amount sufficient to alleviate (for example, ameliorate, reduce, or diminish) at least one of the symptoms associated with a disease condition. For example, a therapeutically effective amount for in vivo injection, that is, for direct injection into the retina, may range from about 10⁶ to about 10¹⁵, for example, from about 10⁸ to about 10¹² genome copies (GC). For in vitro transduction, an effective amount of rAAV virions delivered to cells may range from about 10⁸ to about 10¹³ GC. A therapeutically effective amount of the recombinant AAV vector according to the disclosure may be readily determined by a person of ordinary skill in the art in consideration of the target disease, the age, sex, and height of the subject, the severity of the disease to be treated, and the like.

According to an aspect of the disclosure, the recombinant viral vector is administered in an amount sufficient to generate infection (or transduction) and expression of the heterologous nucleic acid sequence in retinal cells of the subject.

According to an aspect of the disclosure, the retinal cells may be selected from the group consisting of retinal ganglion cells, bipolar cells, photoreceptor cells, Müller glial cells, horizontal cells, amacrine cells, astrocytes, microglial cells, retinal vascular cells, and retinal pigment epithelium (RPE) cells.

According to an aspect of the disclosure, the retinal cells are photoreceptor cells, for example, cone cells and/or rod cells.

According to an aspect of the disclosure, the retinal cells are retinal ganglion cells (RGCs).

According to an aspect of the disclosure, the retinal cells are RPE cells.

According to an aspect of the disclosure, the pharmaceutical composition may be administered one or more times over periods of various intervals, for example, daily, weekly, monthly, yearly, etc. to achieve a desired level of gene expression.

According to an aspect of the disclosure, the pharmaceutical composition may include a pharmaceutically acceptable carrier, excipient, or additive.

According to another aspect of the disclosure, provided is an in vitro method of delivering a heterologous nucleic acid encoding a target product to retinal cells, including contacting the above-described recombinant AAV vector with retinal cells.

According to the disclosure, delivery of a heterologous nucleic acid to retinal cells may be used for treatment of a retinal disease.

According to an aspect of the disclosure, the target product and retinal cells are as described above.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a schematic diagram of an artificial intelligence-based AAV capsid engineering method including a process of training AAV variant screening data by using a deep learning-based model, and designing AAV variants based thereon, in which, specifically, AAV capsid data having delivery capability into retinal cells are collected from human retinal organoids (Data collection), and data on AAV2 capsid sequences and AAV properties such as gene delivery capability into photoreceptor cells are secured through Next-Generation Sequencing (NGS), thereafter, the secured data are preprocessed to be suitable for training of a deep learning model (Data preprocessing), and correlations between sequences and AAV properties are learned through artificial intelligence (Training), next, relationships between amino acids at each position of the sequence and AAV properties are analyzed through the trained model (Analyze), and based thereon, a model capable of predicting AAV2 properties for AAV2 capsid sequences is constructed (Prediction), and using the constructed model, an AAV2 capsid sequence having optimal properties may be designed (Design), and by transducing the designed AAV2 capsid sequence into human retinal organoids and verifying gene delivery capability into photoreceptor cells, an AAV2 variant capsid protein having improved transduction into retinal cells as compared with an AAV having a wild-type AAV2 capsid was selected;

FIG. 2 illustrates a performance evaluation of deep learning models (2D CNN, 1D CNN, and DNN) for photoreceptor tropism for selection of AAV2 variants according to an embodiment, in which, after preprocessing AAV2 capsid sequence data selected from photoreceptor cells by one-hot encoding and training the same, three models based on 2D CNN, 1D CNN, and DNN algorithms, respectively, were constructed, and all three models showed satisfactory training performance, and prediction performance was also confirmed to be satisfactory in parity plots of actual values and predicted values;

FIG. 3 illustrates a vp3 structural image of AAV2.ADN including an ADN variant according to an embodiment predicted by ColabFold, in which the portion indicated by a dotted circle represents an inserted heptameric peptide;

FIG. 4A and FIG. 4B illustrate the gene delivery capability of AAV2.ADN including an ADN variant according to an embodiment, verified in human retinal organoids, in which, after transduction of human retinal organoids (CRX-tdTomato) with recombinant AAV2.ADN (SEQ ID NO: 2), wild-type AAV2 (SEQ ID NO: 1), and improved AAV2.7m8 (SEQ ID NO: 3) and AAV2.NN (SEQ ID NO: 4), each carrying a green fluorescent protein (GFP) gene as a target gene, it was confirmed that AAV2.ADN has superior gene expression efficiency throughout the retinal organoid as compared with wild-type AAV2 and the improved AAV2.7m8 and AAV2.NN, FIG. 4A is a low-magnification image, and FIG. 4B is a high-magnification image;

FIG. 5A and FIG. 5B illustrate the results of quantitative analysis of gene transduction efficiency of AAV2.ADN according to an embodiment, confirmed in human retinal organoids, in which tdTomato fluorescence labels photoreceptor cells, and GFP fluorescence labels cells to which the gene has been delivered, in FIG. 5B, the gene transduction efficiency into all retinal cells (Total) was analyzed as a ratio of GFP-positive cells among total cells, gene transduction efficiency into photoreceptor cells (Photoreceptor) was analyzed as a ratio of GFP-positive cells among tdTomato-positive cells, and gene transduction efficiency into non-photoreceptor retinal cells (Non-photoreceptor) was analyzed as a ratio of GFP-positive cells among tdTomato-negative cells, AAV2.ADN showed significantly higher gene transduction efficiency in photoreceptor cells of retinal organoids than wild-type AAV2 and AAV2.7m8, and showed higher gene transduction efficiency than wild-type AAV2 and the improved AAV2.7m8 and AAV2.NN in total cells and non-photoreceptor cells, respectively, which was statistically significant;

FIG. 6 illustrates the results of immunofluorescence staining showing gene transduction efficiency of recombinant AAV2 vectors carrying a gene encoding GFP as a target gene in AAV2.ADN, wild-type AAV2, improved AAV2.7m8, and AAV2.NN, according to an embodiment, in which antibodies labeling photoreceptor cells and Müller glial cells (CRX and Recoverin antibodies: photoreceptor cells, CARLBP and SOX9: Müller glial cells) and an antibody against GFP fluorescence were used;

FIG. 7 illustrates the results of verifying the in-vivo gene transduction efficiency of AAV2.ADN according to an embodiment, showing expression patterns of a target gene observed after intravitreal administration of AAV2.ADN carrying a tdTomato gene as a target gene, and wild-type AAV2, improved AAV2.7m8, and AAV2.NN as controls, to reporter mice (Nrl-EGFP) in which rod cells are labelled with EGFP fluorescence, wherein, in the figures, RGC refers to retinal ganglion cells, INL refers to the inner nuclear layer, ONL refers to the outer nuclear layer, and OS refers to the outer segment, and while wild-type AAV2 was confirmed to infect RGCs and some INL, AAV2.ADN was confirmed to have significantly higher expression of the delivered tdTomato gene, which was delivered not only to RGCs and INLs but also to the ONL and OS regions, which are the nuclear layers of photoreceptor cells; and

FIG. 8 illustrates the results of quantitative analysis of the in-vivo gene transduction efficiency of AAV2.ADN according to an embodiment, showing expression patterns of a target gene confirmed by FACS analysis after intravitreal administration of AAV2.ADN carrying a tdTomato gene as a target gene, and wild-type AAV2, improved AAV2.7m8, and AAV2.NN as controls, to mice (Nrl-EGFP), in which EGFP fluorescence labels rod cells, and tdTomato fluorescence labels cells to which the gene has been delivered, gene transduction efficiency to all retinal cells (Total) was measured as a ratio of tdTomato-positive cells among total cells, gene transduction efficiency to rod cells was measured as a ratio of tdTomato-positive cells among EGFP-positive cells, and gene transduction efficiency to non-rod retinal cells was measured as a ratio of tdTomato-positive cells among EGFP-negative cells, and AAV2.ADN showed significantly higher gene transduction efficiency in rod cells than wild-type AAV2 and the improved AAV2.7m8 and AAV2.NN, which was statistically significant.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The disclosure will be made clear by reference to the embodiments disclosed in detail below together with the accompanying figures. However, the disclosure is not limited to the examples, but may be embodied in various forms, and the examples are provided merely to make the description of the disclosure complete and to fully convey the scope of the disclosure a person of ordinary skill in the art to which the disclosure pertains, and the disclosure is defined by the claims.

### Example 1. Prediction and Design of AAV Variants Using Artificial Intelligence

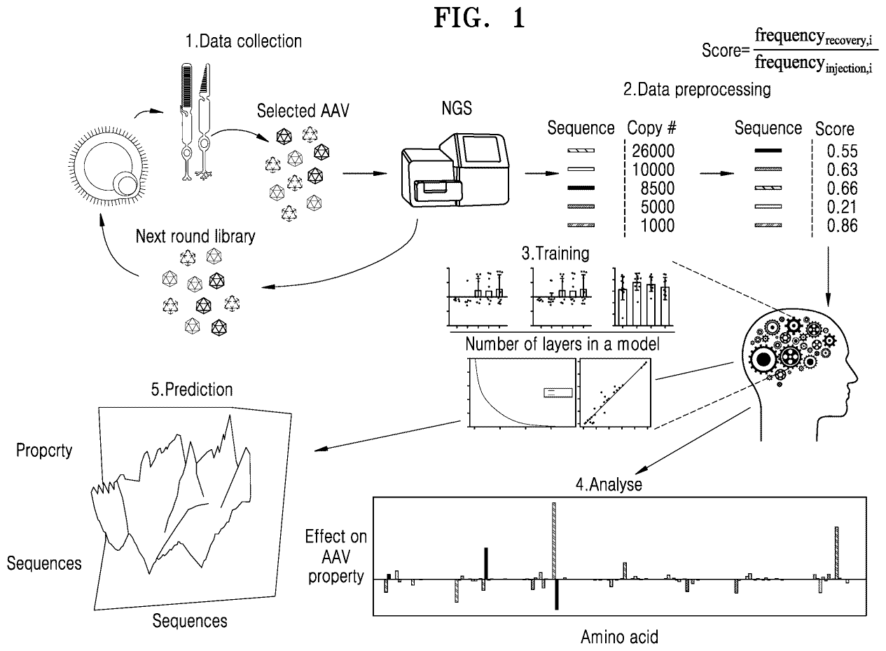

To design and select AAV2 capsid variants capable of efficiently delivering and expressing a target gene to retinal cells, artificial intelligence-based AAV2 capsid engineering was performed. Specifically, to discover AAV capsid sequences that were not experimentally identified in AAV screening using a random library, a deep learning model trained on data selected from human retinal organoids was analyzed using an explainable artificial intelligence (XAI) algorithm, and AAV variants capable of effectively delivering genes to human retinal cells were designed. A schematic diagram of artificial intelligence-based AAV capsid engineering is illustrated in FIG. 1.

AAV capsid data having delivery capability to retinal cells were collected from human retinal organoids, data on AAV2 capsid sequences and AAV properties, for example, gene delivery capability to retinal photoreceptor cells, were secured through NGS, and correlations between AAV capsid sequences and AAV properties such as tropism toward retinal photoreceptor cells were trained through artificial intelligence. Through this, a model for designing AAV capsid sequences capable of predicting AAV capsid sequences having desired properties was constructed. This model analyzes the influence of amino acids at each position of an AAV capsid sequence on AAV properties, and based thereon predicts an AAV capsid sequence having optimal properties. Specifically, by visualizing amino acids affecting AAV properties and their positions, combinations of amino acid compositions and corresponding AAV properties are analyzed to provide predicted values for performance of an AAV capsid sequence, from which an optimal AAV capsid sequence may be selected.

### 1.1. Data Collection by Random Library Screening Using Human Retinal Organoids

### (1) Preparation of In Vitro Human Retinal Organoids

Human retinal organoids to be used for evaluating gene transduction efficacy of AAV2 variants to retinal cells were prepared according to the method disclosed in Wahlin, K. J. et al., Photoreceptor Outer Segment-like Structures in Long-Term 3D Retinas from Human Pluripotent Stem Cells, Sci Rep 7, 766 (2017). Specifically, human retinal organoids were produced using human embryonic stem cells that express tdTomato fluorescent protein specifically in photoreceptor cells. Human embryonic stem cells expressing tdTomato fluorescent protein specifically in retinal cells were cultured in mTesR1 basal medium (Stem Cell Technologies, Vancouver, Canada) on plates coated with Matrigel (Corning, NY, USA). The cells were cultured for 4-5 days prior to induction of differentiation into retinal organoids, and the cultured stem cells were dissociated using Accutase (BD Biosciences, CA, USA), followed by aggregation at a density of 3,000 cells per well using a low-cell adhesion 96-well plate (Corning) having a U-shaped bottom. Thereafter, differentiation and culture were carried out for 30 weeks or more to obtain human retinal organoids. All cultures for the preparation of human retinal organoids were maintained at 37° C. in a humidified environment with 5% carbon dioxide.

### (2) Construction of a Random Library

A random library was constructed by inserting a random heptameric peptide into the GH loop, known as a cellular interaction domain of wild-type AAV2 (including amino acids 587 and 588 of the AAV2 capsid of SEQ ID NO: 1). At this time, the theoretical diversity that the AAV random library may have is 1.28×10⁹, whereas the diversity of the AAV random library that may be practically implemented is 1×10⁶⁻⁷. To overcome such limitations of the AAV random library, artificial intelligence was utilized to train relationships between AAV capsid sequences and human retinal cell targets, thereby designing optimal human retinal cell-targeting AAV variants.

### (3) Selection of Variants Having Gene Delivery Capability to Photoreceptor Cells

### Screening

The prepared human retinal organoids in which photoreceptor cells were labeled with tdTomato fluorescence were transduced with the random library having inserted heptameric peptides at 5×10¹⁰ genome copy (GC), and after 3 weeks, tdTomato fluorescence-based FACS sorting was performed to select variants having high gene transduction efficiency to retinal cells.

### Cell Sorting by FACS and NGS

For fluorescence-activated cell sorting (FACS), human retinal organoids were dissociated into single cells using a Papain Dissociation System (Worthington Biochemical Corporation, NJ, USA). FACS was performed using a BD FACSymphony S6 Cell Sorter (BD Biosciences, CA, USA). In the case of human retinal organoids, tdTomato-positive cells and tdTomato-negative cells were separated.

The separated cells were each collected by centrifugation, and DNA was extracted using a QIAamp DNA Mini Kit (QIAGEN, 51306). PCR was performed to amplify 229 bp including the inserted heptameric peptide from the extracted DNA, and target DNA was obtained as a PCR product using a QIAquick Gel Extraction Kit (QIAGEN, 51306). According to the manufacturer's instructions, Next Generation Sequencing (NGS) analysis of the PCR product was performed using MiSeq Reagent Kits v2 (ILLUMINA, MS-102-2003), thereby collecting AAV capsid data having gene delivery capability to retinal cells. The NGS data included relationships between human retinal cell infection rates according to AAV capsid sequences.

### 1.2. NGS Data Preprocessing for AI Model Training: Mean-Squared Error (MSE)

To design AAV2 variant capsids having gene delivery capability to retinal cells, sequence information of AAV variant capsids that infected human retinal cells obtained through NGS in 1.1 was used. Since AAV capsid sequence information is string data and artificial intelligence may receive only numerical data as input values, a process of converting string-based AAV capsid sequence data into numerical data was required.

For conversion into numerical data, a one-hot encoding technique was utilized, and for one-hot encoding, establishment of a labeling order was required. Training performance was evaluated using two types of labeling orders, one in which amino acids were arranged in alphabetical order and the other in which amino acids were arranged based on their chemical properties. In one-hot encoding in which amino acids were arranged based on chemical properties, lower overfitting and MSE were confirmed, and accordingly, the labeling order was set based on chemical properties (basic, acidic, polar, and nonpolar).

Data obtained from NGS are copy numbers of AAV variant capsid sequences that infected each cell. An AAV variant that occupied a large proportion of the AAV random library used may have a large absolute copy number infecting retinal cells even if its infection efficiency for human retinal cells is low. To correct this, a commonly used log enrichment score (output copy number ratio/input copy number ratio) was introduced and set as a score. The proportion of each individual variant in the AAV library recovered from photoreceptor cells was normalized by the proportion of each individual variant in the injected AAV library. Through this, relative comparison of human photoreceptor cell targeting ability among AAV variants may be made based on changes in the proportion occupied by each individual variant within the AAV library.

### 1.3. Construction of Deep Learning Training Models for Photoreceptor Tropism

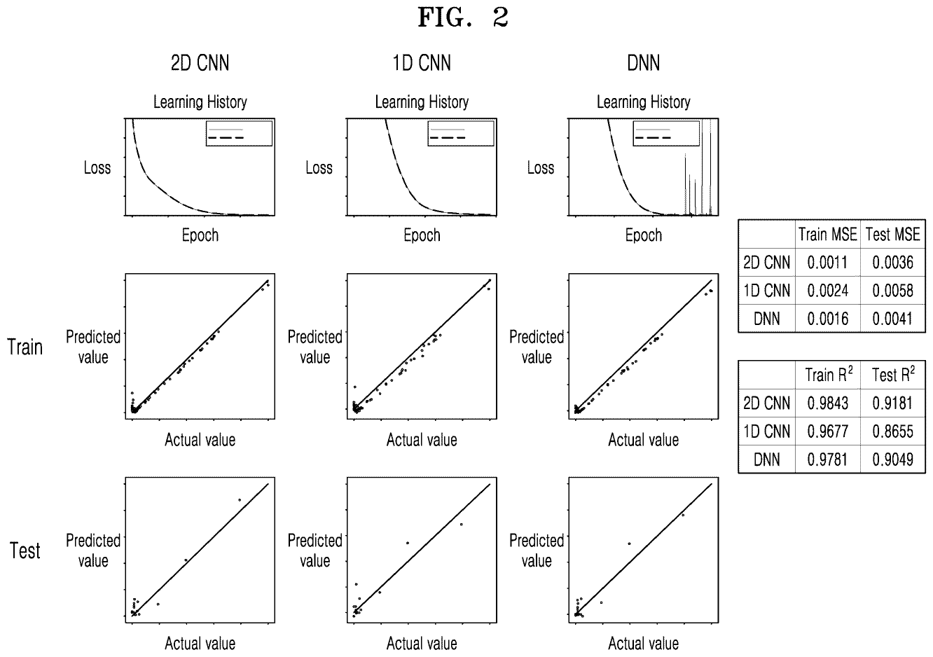

Deep learning models were trained using enrichment scores as output values and recovered AAV capsid sequences as input values. Since abstract characteristics of AAV contained in the AAV capsid sequences needed to be learned, training was performed using deep learning techniques specialized for nonlinearity and abstract information learning. To increase the reliability of training, three deep learning training models were constructed based on 2D CNN, 1D CNN, and DNN algorithms. Based on the data preprocessing method described in 1.2, data selected from photoreceptor cells were preprocessed and then used to train the training models. That is, correlations among AAV2 capsid sequences that reached photoreceptor cells, copy numbers of the sequences, and tropism to photoreceptor cells were trained. All three models showed satisfactory training performance, and it was confirmed through a parity plot (actual value, predicted value) that prediction performance was satisfactory. The results are illustrated in FIG. 2.

The trained deep learning models include weights for 20 types of amino acids at each position of the heptameric peptide with respect to the relationship between AAV capsid sequences and human retinal cell infection rates. Using XAI techniques, the weights trained by the deep learning models were visualized and analyzed to present amino acids capable of effectively infecting human retinal cells. Based on this, an amino acid combination that commonly had a positive effect on human retinal cell infection across the three models was selected, and ‘NAPPKNP’ (hereinafter, AAV2.ADN), which was predicted to have excellent gene delivery capability to photoreceptor cells, was selected. This variant was named the “ADN variant.”

The amino acid sequence (SEQ ID NO: 2) of the capsid protein of AAV2.ADN, in which the ADN variant (indicated in red) is inserted at amino acid position 587 of the AAV2 wild-type capsid protein (SEQ ID NO: 1), is as follows:

MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQA
KKRVLEPLGLVEEPVKTAPGKKRPVEHSPVEPDSSSGTGKAGQQPARKRLNFGQTGDADSVP
DPQPLGQPPAAPSGLGTNTMATGSGAPMADNNEGADGVGNSSGNWHCDSTWMGDRVITTS
TRTWALPTYNNHLYKQISSQSGASNDNHYFGYSTPWGYFDFNRFHCHFSPRDWQRLINNNW
GFRPKRLNFKLFNIQVKEVTQNDGTTTIANNLTSTVQVFTDSEYQLPYVLGSAHQGCLPPFPA
DVFMVPQYGYLILNNGSQAVGRSSFYCLEYFPSQMLRTGNNFTFSYTFEDVPFHSSYAHSQSL
DRLMNPLIDQYLYYLSRTNTPSGTTTQSRLQFSQAGASDIRDQSRNWLPGPCYRQQRVSKTSA
DNNNSEYSWTGATKYHLNGRDSLVNPGPAMASHKDDEEKFFPQSGVLIFGKQGSEKTNVDIE
KVMITDEEEIRTTNPVATEQYGSVSTNLQRGNNAPPKNPRQAATADVNTQGVLPGMVWQDR
DVYLQGPIWAKIPHTDGHFHPSPLMGGFGLKHPPPQILIKNTPVPANPSTTFSAAKFASFITQYS
TGQVSVEIEWELQKENSKRWNPEIQYTSNYNKSVNVDFTVDTNGVYSEPRPIGTRYLTRNL

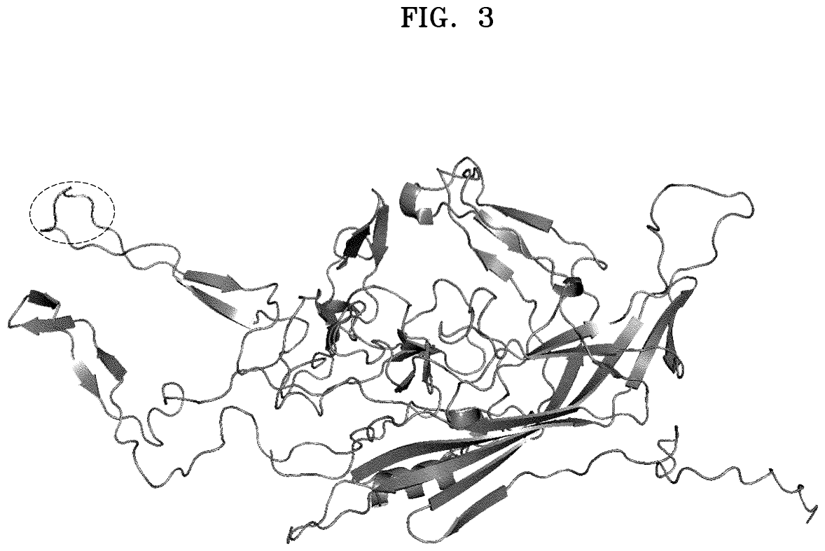

In addition, a vp3 structural image of AAV2.ADN including the ADN variant was confirmed using ColabFold (Nature Methods, 19(6), 679-682). FIG. 3 illustrates a vp3 structural image of AAV2.ADN including an ADN variant according to an embodiment predicted by ColabFold.

### Example 2. In Vitro Validation of AAV2.ADN Variants: Human Retinal Organoids

To verify gene delivery and expression capability of the AAV2.ADN variant to photoreceptor cells selected in Example 1, delivery and expression of a target gene were confirmed in human retinal organoids.

### 2.1. Confirmation of Gene Delivery and Expression by Fluorescence Microscopy

As disclosed in Example 1, retinal organoids including photoreceptor reporters (CRX-tdTomato) that express tdTomato fluorescence specifically in retinal cells were transduced with recombinant vectors AAV2.ADN-EGFP, wild-type AAV2-EGFP, and positive controls AAV2.NN-EGFP and AAV2.7m8-EGFP (see Science Translational Medicine, 5(189), 189ra76 and EMBO Molecular Medicine, 13(4), e13392), each carrying a gene encoding EGFP, at 5×10¹⁰ GC, and gene expression efficiency was confirmed.

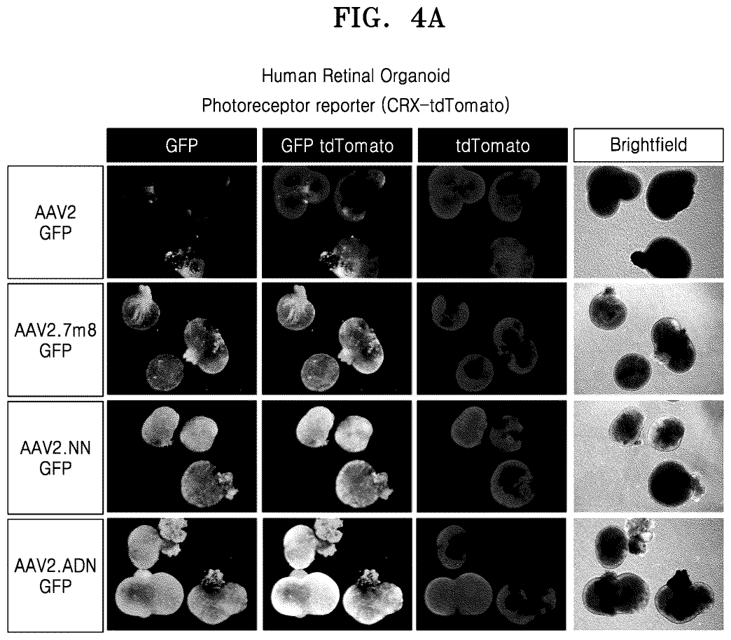

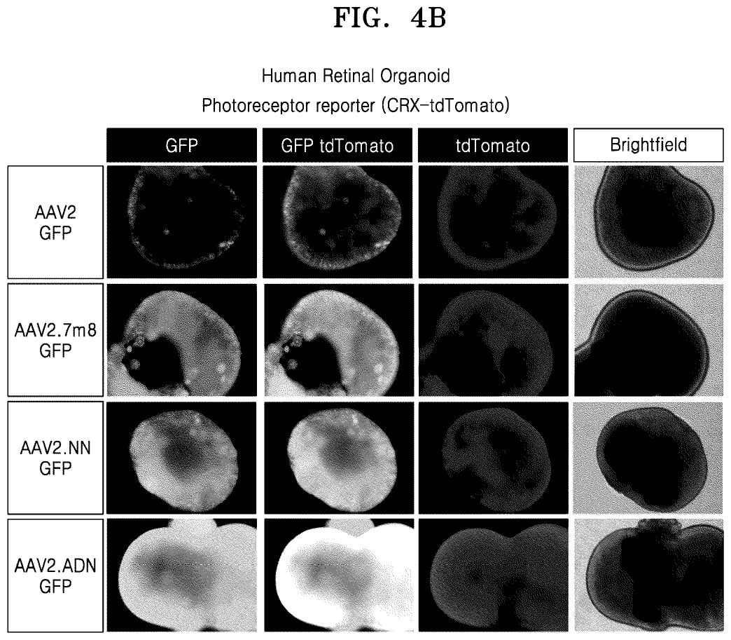

In human retinal organoids, photoreceptor cells, which are target cells of AAV2.ADN, are located at the outermost layer. When a gene expressing EGFP was delivered, it was confirmed that wild-type AAV2, as known in the art, was unable to penetrate the retinal layers and thus failed to infect the interior. In addition, in human retinal organoids treated with AAV2.ADN, regions in which tdTomato fluorescent protein expressed specifically in photoreceptor cells and EGFP fluorescent protein were merged and appeared yellow were broader than those in human retinal organoids treated with AAV2, AAV2.7m8, or AAV2.NN. Accordingly, AAV2.ADN exhibited a significantly higher EGFP gene transduction efficiency throughout the entire human retinal organoid compared to wild-type AAV2 and improved AAV2.7m8 and AAV2.NN. The results are illustrated in FIG. 4A and FIG. 4B.

It was confirmed that the AAV2.ADN variant exhibited superior gene delivery to human photoreceptor cells compared to wild-type AAV2, AAV2.7m8, and AAV2.NN.

### 2.2. Quantitative Analysis of Gene Transduction Efficiency by FACS

Gene expression efficiency and transduction efficiency of AAV2.ADN in human retinal organoids were quantitatively analyzed.

Specifically, human retinal organoids expressing tdTomato fluorescence specifically in photoreceptor cells, prepared as disclosed in 1.1., were transfected with AAV2.ADN carrying a gene encoding EGFP as a target gene, and with control group wild-type AAV2, improved AAV2.7m8, and AAV2.NN, at 5×10¹⁰ vg, and FACS analysis was performed three weeks later. Human retinal organoids were dissociated using a Papain Dissociation System (Worthington Biochemical Corporation, NJ, USA). FACS was performed using a BD FACS Aria II (BD Biosciences, CA, USA), and analysis was conducted using FlowJo software (Tree Star, OR, USA).

tdTomato fluorescence labels photoreceptor cells, and GFP fluorescence labels cells to which the target gene was delivered. Gene transduction efficiency to the entire retinal cells was analyzed as a proportion of GFP-positive cells among total cells, gene transduction efficiency to photoreceptor cells was analyzed as a proportion of GFP-positive cells among tdTomato-positive cells, and gene transduction efficiency to retinal cells other than photoreceptor cells was analyzed as a proportion of GFP-positive cells among tdTomato-negative cells.

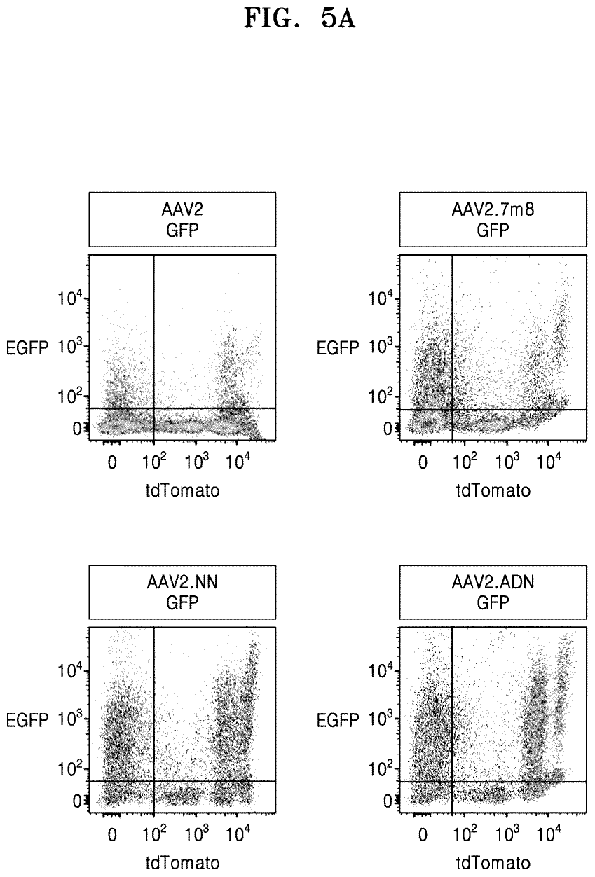

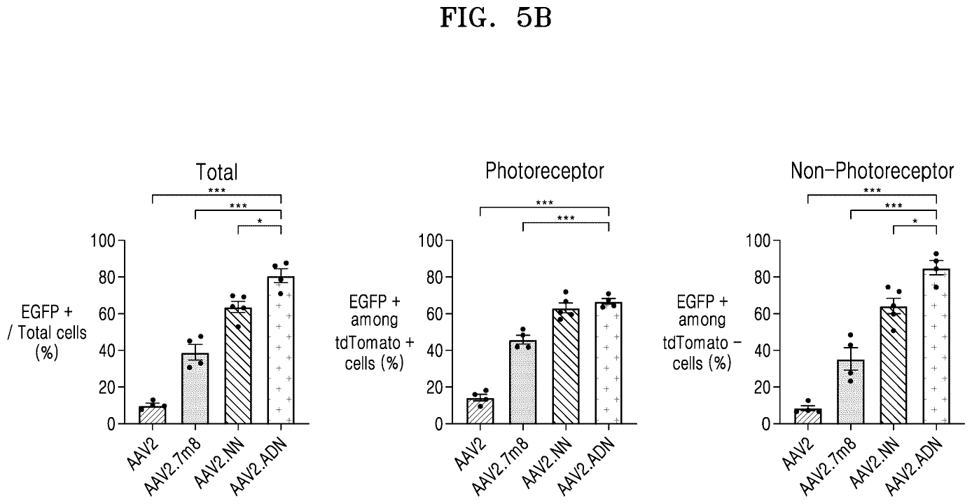

According to FACS analysis, AAV2.ADN exhibited a higher proportion of GFP-positive cells than wild-type AAV2, AAV2.7m8, and AAV2.NN used as control groups, and among these, the proportion of GFP-positive cells among photoreceptor cells (tdTomato-positive) was particularly high. The proportion of GFP-positive cells among total cells in human retinal organoids was 80.88±3.70% for AAV2.ADN, which was significantly higher and statistically significant compared to wild-type AAV2 (10.19±1.09%), improved AAV2.7m8 (39.08±4.26%), and AAV2.NN (63.76±3.05%). In addition, the proportion of GFP-positive cells among photoreceptor cells (tdTomato+) was 66.86±1.68% for the selected AAV2.ADN, which was significantly higher and statistically significant compared to AAV2 (14.31±1.84%) and AAV2.7m8 (46.01±2.37%). Representative FACS analysis images and graphs showing results of repeated FACS analyses are illustrated in FIG. 5A and FIG. 5B, respectively.

### 2.3. Detection of Gene Expression by Immunofluorescence Staining

To confirm gene delivery to photoreceptor cells and Müller glia cells, immunofluorescence staining was performed using antibodies labeling these cells and an antibody against EGFP delivered by AAV2.

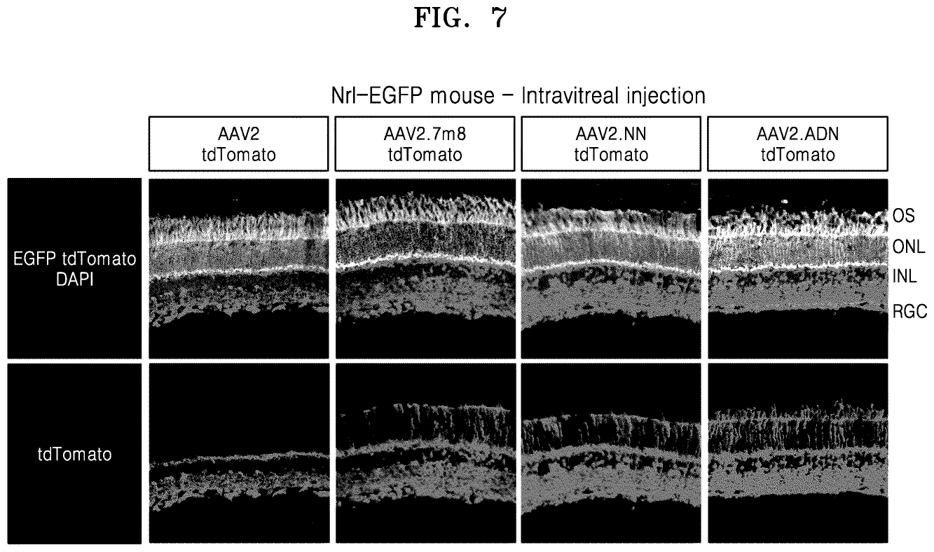

Human retinal organoids transfected with AAV2.ADN-EGFP and control group wild-type AAV2-EGFP, improved AAV2.NN-EGFP, and AAV2.7m8-EGFP were fixed for 25 minutes in a 5% sucrose solution including 4% paraformaldehyde. After fixation, the samples were sequentially treated with 10%, 20%, and 30% sucrose solutions for 1 hour each. For sectioning, human retinal organoids were embedded using an optical coherence tomography (OCT) compound and rapidly frozen using liquid nitrogen vapor. Thereafter, sections having a thickness of 7 μm were prepared using a cryostat (Leica Microsystems, Wetzlar, Germany), and differences in GFP gene expression in the human retinal organoids were examined. The results are illustrated in FIG. 7. Expression of the GFP gene delivered by AAV2 variants and wild-type AAV2 was merged with fluorescence of photoreceptor marker antibodies (CRX and Recoverin antibodies) and fluorescence of Müller glial cell marker antibodies (CRALBP and SOX9 antibodies), respectively. GFP gene expression by the AAV2.ADN variant showed broader merged regions in photoreceptor cells and Müller glia cells than those of wild-type AAV2 and improved AAV2.NN and AAV2.7m8. Accordingly, it was confirmed that the selected AAV2.ADN exhibited higher gene transduction efficiency to photoreceptor cells and Müller glia cells than wild-type AAV2 and improved AAV2.7m8 and AAV2.NN.

### Example 3. In Vivo Validation of AAV2.ADN Variants: Mice

The gene delivery capability of the AAV2.ADN variant to photoreceptor cells selected in Example 1 was confirmed in a reporter mouse (B6.Cg-Tg(Nrl-EGFP)1Asw/J) in which EGFP fluorescence is labeled in rod photoreceptors.

### 3.1. Confirmation of Gene Delivery and Expression by Fluorescence Microscopy

to verify the gene transduction efficiency of AAV2.ADN in mice, AAV2.ADN carrying a gene encoding tdTomato, and as control groups AAV2, AAV2.7m8, and AAV2.NN, were each intravitreally injected at 1×10¹⁰ GC into 8-week-old mice (B6.Cg-Tg(Nrl-EGFP)1Asw/J) expressing EGFP fluorescence in rod cells, and after 3 weeks, the mice were sacrificed and the eyeballs were extracted. The extracted eyeballs were fixed in 1% paraformaldehyde for 1 hour, and thereafter sequentially treated with 10%, 20%, and 30% sucrose solutions for 1 hour each. For sectioning, the eyeballs were embedded using an optical coherence tomography (OCT) compound such that the optic nerve and corneal central junction was horizontally aligned, rapidly frozen using liquid nitrogen vapor, and sectioned to a thickness of 7 μm using a cryostat (Leica Microsystems, Wetzlar, Germany). After staining with DAPI to label cell nuclei, expression of EGFP and tdTomato was imaged using a fluorescence microscope.

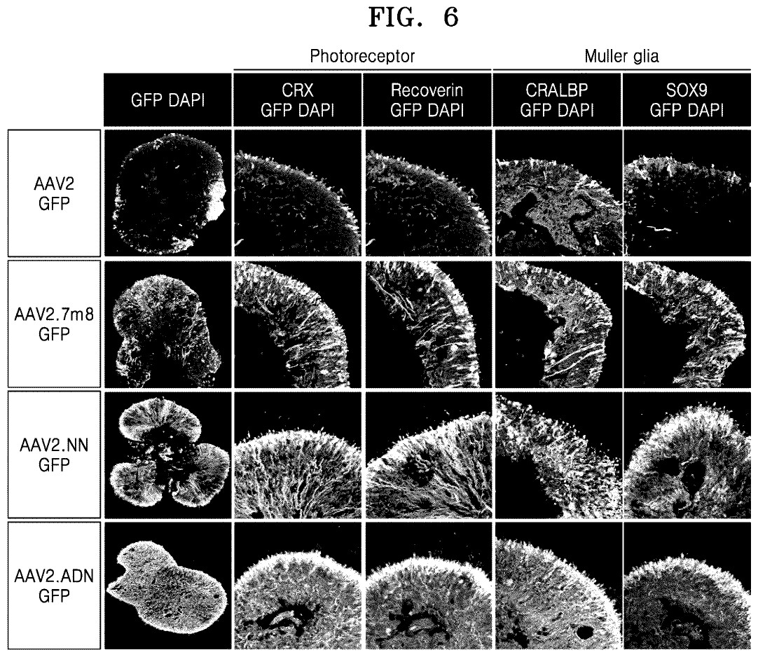

It was confirmed that wild-type AAV2 infected retinal ganglion cells (RGCs) and a portion of the inner nuclear layer (INL). In contrast, AAV2.ADN was confirmed to have significantly higher expression of the tdTomato gene delivered to not only RGCs and INL but also to the outer nuclear layer (ONL) and outer segment (OS) region, which are the nuclear layers of photoreceptor cells. The results are illustrated in FIG. 6.

3.2. Quantitative analysis of gene transduction efficiency by FACS

To quantify the gene transduction efficiency of AAV2.ADN in mice, AAV2.ADN carrying a gene encoding tdTomato, and as control groups AAV2, AAV2.7m8, and AAV2.NN, were each intravitreally injected at 1×10¹⁰ GC into 8-week-old mice (B6.Cg-Tg(Nrl-EGFP)1Asw/J) expressing EGFP fluorescence in rod cells, and after 3 weeks, the mice were sacrificed and the eyeballs were extracted. The retinal tissue was dissociated into single cells using a Papain Dissociation System (Worthington Biochemical Corporation, NJ, USA), and FACS was performed using a BD FACSymphony S6 Cell Sorter (BD Biosciences, CA, USA). FACS analysis was conducted using FlowJo software (Tree Star, OR, USA).

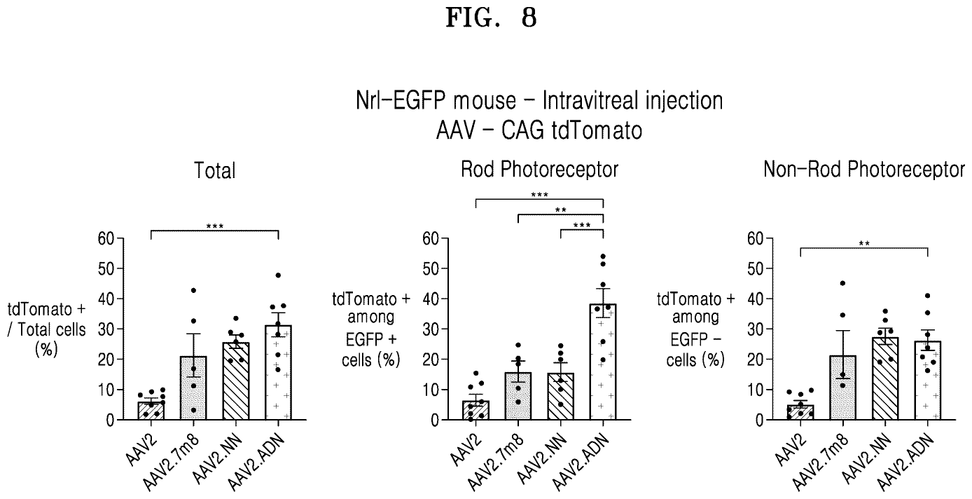

EGFP fluorescence labels rod cells, and tdTomato fluorescence labels cells to which the gene was delivered. The gene transduction efficiency to the entire retinal cells was measured as the proportion of tdTomato-positive cells among total cells, the gene transduction efficiency to rod cells was measured as the proportion of tdTomato-positive cells among EGFP-positive cells, and the gene transduction efficiency to retinal cells excluding rod cells was measured as the proportion of tdTomato-positive cells among EGFP-negative cells. As a result of the FACS analysis, AAV2.ADN exhibited significantly higher gene transduction efficiency in rod cells than wild-type AAV2 and improved AAV2.7m8 and AAV2.NN. The proportion of tdTomato-positive cells among rod cells (EGFP+) was 38.50±4.80% for AAV2.ADN, which was significantly higher than that of AAV2 (6.52±2.00%), AAV2.7m8 (15.95±3.42%), and AAV2.NN (15.78±3.09%), and was statistically significant. In the case of AAV2.ADN, gene transduction efficiency in the entire retinal cells and retinal cells excluding rod cells was higher than that of AAV2 and was statistically significant. The results are illustrated in FIG. 8.

Through these results, it was confirmed that, compared with wild-type AAV2 and improved AAV2.7m8 and AAV2.NN, the ADN variant selected in Example 1 passes through the ILM from the injection site at a high rate via intravitreal injection, delivers a target gene to photoreceptor cells, and expresses the target gene at a high level.

The ADN variant according to the disclosure was confirmed to have excellent gene transduction efficiency through passing through the ILM by intravitreal injection, delivering a target gene to retinal cells including photoreceptors, inducing strong gene expression, providing increased gene expression and lateral spreading compared to wild-type AAV2, and increased photoreceptor-specific gene transduction and gene expression compared to wild-type AAV2 in human retinal organoids. Accordingly, the ADN variant according to the disclosure may be usefully employed in gene therapy for treatment of ocular diseases.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An AAV2 variant capsid protein comprising a peptide NAPPKNP inserted into a GH-loop of a wild-type AAV2 capsid protein.

2. The AAV2 variant capsid protein of claim 1, wherein the peptide is inserted at amino acid position 587 of the wild-type AAV2 capsid protein of SEQ ID NO: 1, or at a corresponding position thereof.

3. The AAV2 variant capsid protein of claim 1, wherein, as compared with a wild-type AAV2 capsid protein, the AAV2 variant capsid protein confers increased infectivity into retinal cells via intravitreal injection.

4. The AAV2 variant capsid protein of claim 3, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.

5. An isolated nucleic acid encoding the AAV2 variant capsid protein of claim 1.

6. A host cell comprising an isolated nucleic acid encoding the AAV2 variant capsid protein of claim 1.

7. A recombinant AAV vector, comprising: the AAV2 variant capsid protein of claim 1; and
a heterologous nucleic acid comprising a sequence encoding a target product.

8. The recombinant AAV vector of claim 7, wherein, as compared with an AAV vector comprising a wild-type AAV2 capsid protein, the recombinant AAV vector provides increased transduction into retinal cells via intravitreal injection.

9. The recombinant AAV vector of claim 8, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.

10. The recombinant AAV vector of claim 7, wherein the target product is a polypeptide or a nucleic acid.

11. A pharmaceutical composition for treatment of an ocular disease, comprising the recombinant AAV vector of claim 7.

12. The pharmaceutical composition of claim 11, wherein the ocular disease is selected from retina-vitreous-choroid related diseases, retinal vascular diseases, macular diseases, hereditary retinal diseases, hereditary vitreous diseases, hereditary choroidal diseases, intraocular inflammatory diseases, intraocular tumors, glaucoma, and optic neuropathies.

13. The pharmaceutical composition of claim 11, wherein the ocular disease is selected from macular degeneration, diabetic retinopathy, occlusion of retinal vessels, macular telangiectasia, retinopathy of prematurity, myopic degeneration, retinitis pigmentosa, Leber congenital amaurosis, Best's Disease, Stargardt's Disease, congenital stationary night blindness, X-linked retinoschisis, Bietti crystalline dystrophy, achromatopsia, cone dystrophy, cone-rod dystrophy, maculopathy, Usher syndrome, Bardet-Biedl syndrome, syndromic retinitis pigmentosa, pan choroidal atrophy, central areolar choroidal dystrophy, gyrate atrophy of choroid and retina, glaucoma, optic neuropathy, uveitis, uveal melanoma, intraocular lymphoma, retinoblastoma, retinoschisis, and retinal injury.

14. The pharmaceutical composition of claim 11, wherein the pharmaceutical composition is administered by intravitreal injection, subretinal injection, or suprachoroidal injection.

15. An in-vitro method of delivering a heterologous nucleic acid into retinal cells, comprising contacting the retinal cells with the recombinant AAV vector of claim 7.

16. The method of claim 15, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.


SEQUENCE LISTING (4 sequences)

SEQ ID NO: 1 - protein, 735 aa, Adeno-associated dependoparvovirus A [sequence omitted]

SEQ ID NO: 2 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNNAPPKNPRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAANNPTPSRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL